(19) United States
(12) Patent Application Publication
(10) Pub. No.: US 2017/0049716 A1
(43) Pub. Date: Feb. 23, 2017

(54) NITISINONE DOSING REGIMENS FOR THE TREATMENT OF ALKAPTONURIA

(71) Applicant: Swedish Orphan Biovitrum International AB, Stockholm (SE)

(72) Inventor: Birgitta Olsson, Stenhamra (SE)

(73) Assignee: Swedish Orphan Biovitrum International AB, Stockholm (SE)

(21) Appl. No.: 15/307,622

(22) PCT Filed: Apr. 29, 2015
(86) PCT No.: PCT/EP2015/059352

§ 371 Date: Oct. 28, 2016

(30) Foreign Application Priority Data
Apr. 30, 2014 (SE) 1450521-8

(51) Int. Cl. A61K 31/122; A61K 9/00

(52) U.S. Cl. CPC A61K 31/122; A61K 9/0053

(57) ABSTRACT

The invention relates to nitisinone (2-[2-nitro-4-(trifluoromethyl)benzoyl]-1,3-cyclohexanedione) for use in the treatment of alkaptonuria, wherein nitisinone is administered in a dose of at least 4 mg per day. The invention also relates to a pharmaceutical composition comprising nitisinone for use in the treatment of alkaptonuria, wherein nitisinone is administered in a dose of at least 4 mg per day.

### TECHNICAL FIELD

The invention relates to nitisinone (2-[2-nitro-4-(trifluoromethyl)benzoyl]-1,3-cyclohexanedione) for use in the treatment of alkaptonuria, wherein nitisinone is administered in a dose of at least 4 mg per day. The invention also relates to a pharmaceutical composition comprising nitisinone for use in the treatment of alkaptonuria, wherein nitisinone is administered in a dose of at least 4 mg per day.

### BACKGROUND ART

Alkaptonuria (AKU) is an autosomal recessive disorder caused by a deficiency of the enzyme homogentisate 1,2-dioxygenase (HGD). It is a rare disease affecting approximately one in every 250,000 to 1 million people. Due to the absence of HGD, alkaptonuria patients are unable to fully metabolize the amino acid tyrosine, which results in high plasma (or serum) levels of homogentisic acid (HGA). Despite efficient and marked urinary excretion of much of the HGA formed in AKU patients, some of it is oxidized to a melanin-like polymeric pigment via benzoquinone acetic acid (BQA). This pigment polymer is deposited in connective tissues (particularly cartilage) in a process termed ochronosis. This leads to severe arthritis of the spine and synovial joints with an early onset. Until the late 20s or early 30s, there are few clinical features aside from dark urine. Thereafter, progressive arthritic pain begins, affecting the spine and all synovial joints. The high levels of HGA further cause damage to heart valves and lead to the formation of kidney stones, as well as prostate stones in men.

There is currently no approved pharmacological treatment available for lowering HGA in patients with AKU and treatment options are limited to treatment of the disease sequelae as they arise, including physiotherapy, surgery and analgesia. Treatment with vitamin C, to inhibit the oxidative conversion of HGA to melanin-like polymeric pigment, has not proven helpful (La Du. Alkaptonuria. In: Scriver, Beaudet, Sly, Valle and Vogelstein (eds), The Metabolic and Molecular Bases of Inherited Disease. McGraw-Hill, New York, 2001, vol. 2, p. 2109-2123). Dietary therapy restricting the intake of phenylalanine and tyrosine has not shown to be effective in sufficiently reducing HGA in adults and has had no demonstrable efficacy in improving the symptoms of AKU (de Haas et al., J. Inherit. Metab. Dis. 1998, vol. 21, no. 8, p. 791-798). Such dietary restrictions are furthermore difficult to maintain.

Nitisinone has been shown to reduce plasma/serum HGA levels and urinary excretion in patients with AKU (Phornphutkul et al., N. Engl. J. Med. 2002, vol. 347, no. 26, p.2111-2121; Suwannarat et al., Metabolism 2005, vol. 54, p.719-728; Introne et al., Mol. Genet. Metab. 2011, vol. 103, no. 4, p.307-314).

Nitisinone (2-[2-nitro-4-(trifluoromethyl)benzoyl]-1,3-cyclohexanedione) is a competitive inhibitor of the enzyme 4-hydroxyphenylpyruvate dioxygenase (HPPD). Under the brand name Orfadin®, it is used in the treatment of hypertyrosinaemia type 1 (HT-1) where it acts by blocking the metabolic degradation of 4-hydroxyphenylpyruvate. Nitisinone thereby prevents the formation of HGA and the accumulation of the toxic intermediates maleylacetoacetate (MAA) and fumarylacetoacetate (FAA) in HT-1 patients (see scheme 1).

Nitisinone has also been investigated for the treatment of AKU in a long-term (36 months) clinical trial in a dose of 2 mg/day. Although the urinary excretion of nitisinone was reduced by about 95% on average, compared to pre-treatment levels, the trial failed to show an effect on the primary efficacy variable, hip rotation (Introne et al., Mol. Genet. Metab. 2011, vol. 103, no. 4, p. 307-314).

There is therefore a continued need for improved treatment of AKU.

### BRIEF DESCRIPTION OF THE DRAWINGS

FIG. 1 shows a plot of the urinary excretion of HGA at baseline and at week 4 for all patients (including untreated controls).

FIG. 2 shows a plot of u-HGA₂₄ (μmol) at week 4 for nitisinone-treated patients.

FIG. 3 shows a plot of the daily average serum concentrations of tyrosine (μmol/L) at baseline and week 4 for nitisinone-treated patients.

FIG. 4 shows a plot of the urinary excretion of HGA and serum concentrations of tyrosine at week 4 for all patients (including untreated controls).

FIG. 5 shows a plot of the urinary excretion of HGA and serum concentrations of tyrosine at week 4 for nitisinone-treated patients.

FIG. 6 shows the relationship between average daily serum tyrosine concentrations and urinary excretion of HGA for nitisinone-treated patients (1-8 mg daily).

FIG. 7 shows the relationship between average daily serum tyrosine concentrations and urinary excretion of HGA for nitisinone-treated patients (2-8 mg daily). Data for one patient on 2 mg nitisinone, with u-HGA₂₄=3484 μmol and s-Tyr=596 μmol/L, is not shown.

### DISCLOSURE OF THE INVENTION

The inevitable consequence of treatment with nitisinone is the elevation of tyrosine levels. In the treatment of HT-1, it is therefore recommended that a more restricted tyrosine and phenylalanine diet should be implemented to keep plasma tyrosine levels below 500 μmol/L. In a 3-year study of nitisinone in alkaptonuria, where no diet restrictions were applied, tyrosine levels in untreated patients were approximately 60 μM, whereas the 20 patients on nitisinone 2 mg daily had tyrosine levels of 332-1528 μM, with an average of 800 μM (Introne et al., supra). Stable but variably increased plasma tyrosine concentrations were seen by 3-4 weeks post-nitisinone. It is thus difficult to keep the tyrosine concentrations at or below an acceptable level without diet restrictions.

One of the clinical consequences of high serum tyrosine levels is the precipitation of tyrosine crystals in the eye, leading to corneal irritation or pain. Patients may also experience vision problems such as blurred or impaired vision. Although these symptoms disappear after discontinuation of the treatment, they recur when treatment is restarted. Another clinical consequence of the high serum tyrosine concentrations is the negative effect on cognitive function. In a recent study (Bendadi et al., J. Pediatr. 2014, vol. 164, no. 2, p.398-401), it was found that the IQ of children treated with nitisinone was considerably lower than their unaffected siblings. Another study (Masurel-Paulet et al., J. Inherit. Metab. Dis. 2008, vol. 31, no. 1, p. 81-87) reported cognitive impairment causing schooling difficulties in eight out of 23 school-age patients with HT-1 that were treated with nitisinone. In six out of these eight patients major cognitive disturbances (memory and concentration difficulties, slowness) were noted.

In view of these side effects, it is of great importance that the serum tyrosine levels are kept as low as possible. In healthy individuals, serum tyrosine levels are normally between about 40 and 90 μmol/L, and levels higher than 500 μmol/L should generally be avoided. However, at a nitisinone dose of only 2 mg/day, serum tyrosine levels already are between 600 and 1000 μmol/L.

The increase in serum tyrosine levels is a consequence of the inhibition of HPPD with nitisinone (see scheme 1). It was therefore assumed that any decrease in serum or urine HGA would be mirrored by a simultaneous increase of serum tyrosine. With respect to the high serum tyrosine levels observed at a nitisinone dose of only 2 mg/day, it was thus believed that it would not be possible to use higher doses of nitisinone as this would cause even higher tyrosine levels and an increased risk of serious side effects related to tyrosinaemia.

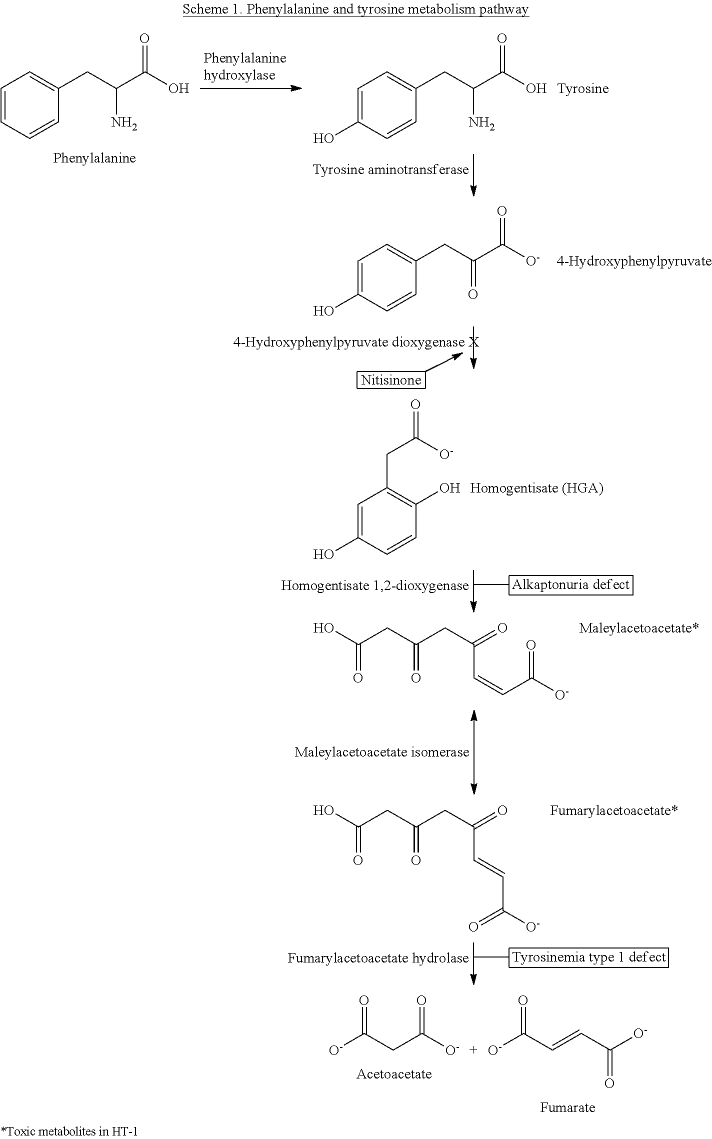

During the study that forms the basis for this invention, it has been found that with increasing doses of nitisinone, the urinary excretion of HGA during 24 hours (u-HGA₂₄) decreased in a clear dose-related manner. However, despite a slight increase in mean serum concentrations of tyrosine (s-Tyr) with increasing doses of nitisinone, the dose-response relationship for tyrosine was less distinct than what was seen for the urinary excretion of HGA. With one exception, a nitisinone dose of 2 mg or higher per day resulted in u-HGA₂₄ values of less than 2000 μmol. No correlation could be seen between s-Tyr and u-HGA₂₄ values when the latter was decreased to below 2000 μmol.

It has thus surprisingly been discovered that administration of nitisinone at considerably higher doses than previously used in the treatment of alkaptonuria may suppress the formation of HGA to more than 99%, while only marginally further increasing the tyrosine level in serum. In other words, the dose of nitisinone can be increased without an increased risk of for example tyrosine-related side-effects.

Thus, in a first aspect, the invention relates to nitisinone for use in the treatment of alkaptonuria, wherein nitisinone is administered in a dose of at least 4 mg per day.

The daily dose of nitisinone as disclosed herein represents a fixed daily dose. This means that the dose is not adjusted based on e.g. body weight of the patient. A dose of at least 4 mg nitisinone per day thus represents the minimum level of a fixed daily amount of nitisinone administered to a patient.

In a preferred embodiment of the invention, nitisinone is administered in a dose of at least 6 mg per day. In a more preferred embodiment, nitisinone is administered in a dose of at least 8 mg per day. In a yet more preferred embodiment, nitisinone is administered in a dose of at least 10 mg per day.

It has been found that administration of nitisinone in a dose of 8 mg per day decreases the urinary excretion of HGA up to more than >99%. It is estimated that a dose of 10 mg per day should be sufficient to suppress the formation of HGA almost completely. Thus, in another preferred embodiment, nitisinone is administered in a dose of 8-12 mg per day. In a most preferred embodiment, nitisinone is administered in a dose of 10 mg per day.

In order to limit the risk of side effects, the dose of nitisinone should not exceed 15 mg per day. It can be estimated by extrapolation that such a dose should be sufficient to decrease u-HGA by about 99.9% in a majority of patients. Thus, in one embodiment, nitisinone is administered in a dose of at least 4 and up to 15 mg per day. In another embodiment, nitisinone is administered in a dose of at least 6 and up to 15 mg per day. In yet another embodiment, nitisinone is administered in a dose of at least 8 and up to 15 mg per day. In yet another embodiment, nitisinone is administered in a dose of at least 10 and up to 15 mg per day.

The invention also relates to the use of nitisinone in the manufacture of a medicament for the treatment of alkaptonuria, wherein the nitisinone is administered in the doses described above. The invention also relates to a method of treating alkaptonuria, comprising administering nitisinone to a patient in need of such treatment in the doses described above.

In another embodiment, the invention relates to nitisinone for use in the treatment of alkaptonuria as disclosed herein, wherein nitisinone is administered as a pharmaceutical composition which comprises nitisinone in admixture with one or more pharmaceutically acceptable excipients.

In another aspect, the invention relates to a pharmaceutical composition comprising nitisinone for use in the treatment of alkaptonuria, wherein nitisinone is administered in a dose of at least 4 mg per day. In a preferred embodiment, nitisinone is administered in a dose of at least 6 mg per day. In a more preferred embodiment, nitisinone is administered in a dose of at least 8 mg per day. In a most preferred embodiment, nitisinone is administered in a dose of 8-12 mg per day, such as 10 mg per day. In another embodiment, nitisinone is administered in a dose of at least 4 and up to 15 mg per day. In another embodiment, nitisinone is administered in a dose of at least 6 and up to 15 mg per day. In yet another embodiment, nitisinone is administered in a dose of at least 8 and up to 15 mg per day. In yet another embodiment, nitisinone is administered in a dose of at least 10 and up to 15 mg per day. The pharmaceutical composition comprising nitisinone may additionally comprise one or more pharmaceutically acceptable excipients.

The daily dose of nitisinone may be a single dose that is administered once a day, or may be divided into two or more smaller doses that are administered several times a day. The frequency of administration can remain constant or be variable during the duration of the treatment. Preferably, nitisinone is administered once daily as a single dose.

In one embodiment, nitisinone is administered orally.

Nitisinone can be administered in a composition comprising a therapeutically effective amount of the active ingredient, in admixture with one or more pharmaceutically acceptable excipients. A composition comprising nitisinone can be formulated as, e.g., capsules, tablets, solutions and oral dispersions. A suitable liquid pharmaceutical composition for oral administration is disclosed in WO 2012/177214. Said liquid pharmaceutical composition comprises a suspension of an effective amount of micronized nitisinone and citric acid buffer having a pH in the range of 2.5 to 3.5, preferably pH 3.0.

The invention is further illustrated by means of the following examples, which do not limit the invention in any respect. All cited documents and references are incorporated herein by reference.

Abbreviations

AKU alkaptonuria

Cₐᵥ average serum concentrations over the 24-hour dosage interval

FAA fumarylacetoacetate

FAH fumarylacetoacetate hydrolase

HGA homogentisic acid

HGD homogentisate 1,2-dioxygenase

HPPA 4-hydroxyphenylpyruvic acid

HPPD 4-hydroxyphenylpyruvate dioxygenase

HT-1 hereditary hypertyrosinaemia type 1

LLOQ lower limit of quantitation

MAA maleylacetoacetate

s-HGA serum concentrations of HGA

s-Tyr serum concentrations of tyrosine

u-HGA₂₄ urinary excretion of HGA during 24 hours

### EXAMPLES

### Example 1

Dose-Response Study of Nitisinone

Methods

Study Design

A randomized, open-label, parallel-group dose-response study with a no-treatment control group was performed. Patients with AKU were randomized to receive either 1 mg, 2 mg, 4 mg or 8 mg nitisinone once daily (oral administration) or no treatment (control). Forty patients were randomized, equally distributed amongst the five groups (8 patients per group).

Patients

Inclusion Criteria

A patient had to fulfill the following criteria in order to be included in the study:
Diagnosis of AKU verified by documented elevated urinary homogentisic acid excretion.
Age ≧18 years.
Willing and able to visit the investigational site for study visits.
Signed written informed consent given.

Exclusion Criteria

The presence of any of the following excluded a patient from inclusion in the study:
Currently pregnant or lactating.
Female patient of child-bearing potential not using a reliable method of contraception.
Known allergy to nitisinone or any of the constituents of the investigational product.
Current keratopathy or uncontrolled glaucoma.
Current malignancy.
Uncontrolled hypertension (blood pressure greater than 180 mmHg systolic or greater than 95 mmHg diastolic).
Unstable cardiovascular disease.
Serum potassium <3.0 mmol/L.
eGFR <60 mL/min.
ALT >3× upper limit of normal.
Hemoglobin <10.0 g/dL.
Platelets <100×10⁹/L.
White blood count <3.0×10⁹/L.
History of alcohol or drug abuse.
Participation in another clinical study within 3 months of randomization.
Treatment with nitisinone within 60 days of randomization.
Psychiatric illness or neurological disease that interferes with compliance or communication with health care personnel.
Foreseeable inability to cooperate with given instructions or study procedures.
Any other medical condition which in the opinion of the investigator makes the patient unsuitable for inclusion.

Treatments

Nitisinone was administered as an oral suspension containing 4 mg/mL. The following dose volumes were administered once daily, in the morning:

| | |
|---|---|
| 1 mg | 0.25 mL |
| 2 mg | 0.50 mL |
| 4 mg | 1.00 mL |
| 8 mg | 2.00 mL |

Each dose was given to a group of 8 patients, and there was an untreated control group also consisting of 8 patients.

Assessments

Urinary HGA

Urinary excretion of HGA over a 24-hour period (u-HGA₂₄) was assessed at weeks 0, 2 and 4. Urine was collected into 2.5-L bottles containing 30 mL of 5N H₂SO₄. The exact length of the collection interval and the volume of the collected urine were recorded. The concentration of HGA in the urine was measured by liquid chromatography tandem mass spectrometry (LCMSMS). The 24-hour excretion of HGA was calculated by multiplying the concentration with the volume of the collected urine, and correcting for any deviation from a 24-hour collection period. (There were no reports of missed samples within the collection interval.)

Serum Tyrosine and HGA

Measurements of serum HGA (s-HGA) and tyrosine (s-Tyr) concentrations were performed at weeks 0, 2 and 4. At all visits a full 24-hour profile was determined at the following time-points: pre-dose (immediately after breakfast) and 0.5, 1,2, 3, 4, 6, 8, 10, 12, 15, 18,24 hours post-dose.

The concentrations of HGA and tyrosine in the serum were measured by liquid chromatography tandem mass spectrometry (LCMSMS). The lower limit of quantification (LLOQ) for HGA was 3.1 μmol/L.

Maximum and daily average serum concentrations of HGA and tyrosine were determined. The daily average concentrations were determined by dividing the area under the concentration vs. time curves, calculated by means of the trapezoidal rule, by 24.

Statistics

All data are presented with descriptive statistics, including mean, standard deviation, median, minimum and maximum values.

Results

Demographics and Baseline Characteristics

Patient baseline data are presented in Table 1.

TABLE 1
Demographics and baseline characteristics

| | | Untreated | 1 mg | 2 mg | 4 mg | 8 mg | Total |
|---|---|---|---|---|---|---|---|
| | | (N = 8) | (N = 8) | (N = 8) | (N = 8) | (N = 8) | (N = 40) |
| Age | n | 8 | 8 | 8 | 8 | 8 | 40 |
| (years) | Mean | 45.9 | 44.4 | 43.9 | 47.3 | 54.4 | 47.2 |
| | SD | 15.25 | 10.93 | 13.71 | 10.74 | 7.29 | 11.91 |
| | Median | 54.5 | 45.5 | 47.0 | 49.0 | 57.0 | 50.0 |
| | min | 19 | 30 | 19 | 28 | 40 | 19 |
| | max | 58 | 58 | 61 | 60 | 62 | 62 |
| Body | n | 8 | 8 | 8 | 8 | 8 | 40 |
| weight | Mean | 71.0 | 86.9 | 74.6 | 76.9 | 81.1 | 78.1 |
| (kg) | SD | 23.49 | 15.91 | 10.91 | 14.30 | 13.65 | 16.33 |
| | Median | 71.0 | 80.5 | 72.0 | 75.5 | 77.5 | 75.0 |
| | min | 40 | 69 | 59 | 61 | 70 | 40 |
| | max | 116 | 111 | 91 | 107 | 112 | 116 |
| Height | n | 8 | 8 | 8 | 8 | 8 | 40 |
| (cm) | Mean | 165.3 | 170.6 | 167.1 | 168.4 | 165.9 | 167.5 |
| | SD | 12.09 | 7.09 | 9.36 | 5.90 | 6.71 | 8.31 |
| | Median | 166.5 | 171.5 | 166.0 | 167.5 | 167.0 | 168.0 |
| | min | 143 | 161 | 154 | 162 | 155 | 143 |
| | max | 182 | 180 | 180 | 179 | 173 | 182 |
| Sex | Female | 4 (50.0%) | 1 (12.5%) | 3 (37.5%) | 3 (37.5%) | 2 (25.0%) | 13 (32.5%) |
| n (%) | Male | 4 (50.0%) | 7 (87.5%) | 5 (62.5%) | 5 (62.5%) | 6 (75.0%) | 27 (67.5%) |

Overall, there were 13 females and 27 males participating in the study. All included patients completed the study and there were no protocol deviations affecting the results of the study.

Urinary Excretion of HGA During 24 Hours (u-HGA)

Urinary excretion of HGA at baseline and week 4 are presented in Table 2. The relative decrease in u-HGA₂₄ is presented in Table 3.

TABLE 2
u-HGA₂₄ (μmol/24h) at baseline and week 4

| | | Untreated | 1 mg | 2 mg | 4 mg | 8 mg |
|---|---|---|---|---|---|---|
| Visit | Statistic | (N = 8) | (N = 8) | (N = 8) | (N = 8) | (N = 8) |
| Week 0 | n | 8 | 8 | 8 | 8 | 8 |
| | Mean | 29835.8 | 36757.0 | 31439.8 | 35453.4 | 27353.5 |
| | SD | 5067.04 | 14614.41 | 7388.00 | 13580.94 | 5234.89 |
| | Median | 29041.3 | 34034.4 | 31754.7 | 31502.7 | 28728.7 |
| | min | 22270.1 | 20743.4 | 19994.2 | 14442.9 | 17075.1 |
| | max | 38832.2 | 69502.9 | 40747.2 | 55393.9 | 32896.6 |
| Week 4 | n | 8 | 8 | 8 | 8 | 8 |
| | Mean | 31041.8 | 3931.0 | 1602.5 | 733.8 | 146.3 |
| | SD | 4603.44 | 1707.14 | 841.65 | 430.29 | 51.18 |
| | Median | 32535.5 | 3415.6 | 1515.6 | 790.5 | 140.4 |
| | min | 22247.4 | 1847.6 | 627.0 | 164.2 | 83.1 |
| | max | 35436.3 | 6752.8 | 3484.1 | 1478.4 | 223.0 |

TABLE 3
Relative decrease in u-HGA₂₄ (%) in nitisinone-treated patients
and untreated controls from baseline to week 4

| | Untreated | 1 mg | 2 mg | 4 mg | 8 mg |
|---|---|---|---|---|---|
| | (N = 8) | (N = 8) | (N = 8) | (N = 8) | (N = 8) |
| Mean | −5.64 | 88.72 | 94.78 | 98.15 | 99.41 |
| SD | 19.28 | 4.99 | 2.64 | 0.82 | 0.34 |
| Median | −2.69 | 87.99 | 95.64 | 98.19 | 99.52 |
| Min | −43.23 | 4.91 | 89.08 | 96.74 | 98.69 |
| Max | 13.69 | 91.05 | 96.86 | 99.41 | 99.70 |

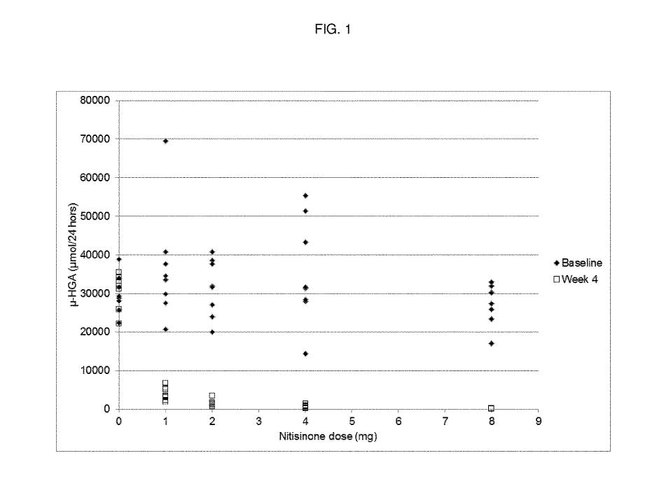

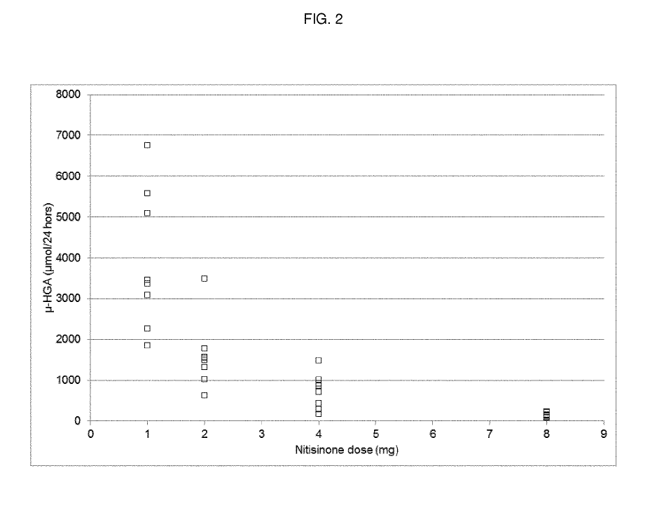

The results are presented graphically in FIG. 1 (all patients, including the untreated control group) and, for better resolution, in FIG. 2 (nitisinone-treated patients only). As can be seen from the tables and figures, treatment with nitisinone led to a dose-dependent decrease in urinary excretion of HGA, and the inter-individual variability in the data also decreases with increasing doses.

Serum HGA

A total of 14 patients had HGA concentrations below the lower limit of quantification (3.1 mmol/L) in all samples and it was therefore not possible to calculate all statistics for this variable, nor to include the data for these 14 individuals in a graph. The average serum concentrations of HGA, over a 24-hour sampling period at baseline and week 4 are presented, as far as possible in Table 4.

TABLE 4
Daily average serum concentrations of HGA (μmol/L) in nitisinone-treated patients
and untreated controls at baseline and week 4

| | | Untreated | 1 mg | 2 mg | 4 mg | 8 mg |
|---|---|---|---|---|---|---|
| Visit | Statistic | (N = 8) | (N = 8) | (N = 8) | (N = 8) | (N = 8) |
| Week 0 | n | 8 | 8 | 8 | 8 | 8 |
| | Mean | 36.7 | 34.2 | 36.6 | 38.5 | 36.6 |
| | SD | 14.0 | 7.0 | 10.7 | 8.2 | 8.2 |
| | Median | 34.6 | 33.0 | 34.2 | 37.7 | 38.6 |
| | min | 19.5 | 25.5 | 25.1 | 28.9 | 25.2 |
| | max | 66.7 | 44.1 | 59.1 | 49.9 | 47.7 |
| Week 4 | n | 8 | 8 | 8 | 8 | 8 |
| | Mean | 35.3 | 4.1 | ND | ND | ND |
| | SD | 12.1 | 3.1 | ND | ND | ND |
| | Median | 36.7 | 3.9 | <3.1 | <3.1 | <3.1 |
| | min | 21.0 | 3.1 | <3.1 | <3.1 | <3.1 |
| | max | 52.0 | 7.3 | 5.8 | <3.1 | 0.4 |

The data in Table 4 do not allow any definite conclusion to be drawn regarding the dose-response relationship for serum HGA, since many patients had concentrations below the LLOQ. However, the increasing number of patients with HGA concentrations below the LLOQ indicates that there was a dose-response relationship also for this variable. There were 3 patients on 2 mg, 4 on 4 mg and 7 on 8 mg with no quantifiable concentrations in any of the samples collected over the 24-hour dosage interval.

Serum Tyrosine

The average serum concentrations of tyrosine, measured over a 24-hour period, are presented for baseline and week 4 in Table 5.

TABLE 5
Daily average serum concentrations of tyrosine (μmol/L) in nitisinone-treated patients
and untreated controls at baseline and week 4

| | | Untreated | 1 mg | 2 mg | 4 mg | 8 mg |
|---|---|---|---|---|---|---|
| Visit | Statistic | (N = 8) | (N = 8) | (N = 8) | (N = 8) | (N = 8) |
| Week 0 | n | 8 | 8 | 8 | 8 | 8 |
| | Mean | 60 | 56 | 64 | 60 | 60 |
| | SD | 10 | 4 | 12 | 10 | 10 |
| | Median | 60 | 56 | 63 | 58 | 59 |
| | min | 45 | 50 | 46 | 47 | 50 |
| | max | 80 | 61 | 79 | 75 | 82 |
| Week 4 | n | 8 | 8 | 8 | 8 | 8 |
| | Mean | 61 | 670 | 734 | 800 | 856 |
| | SD | 10 | 110 | 125 | 126 | 107 |
| | Median | 58 | 667 | 702 | 806 | 860 |
| | min | 52 | 511 | 596 | 639 | 627 |
| | max | 83 | 879 | 948 | 1059 | 970 |

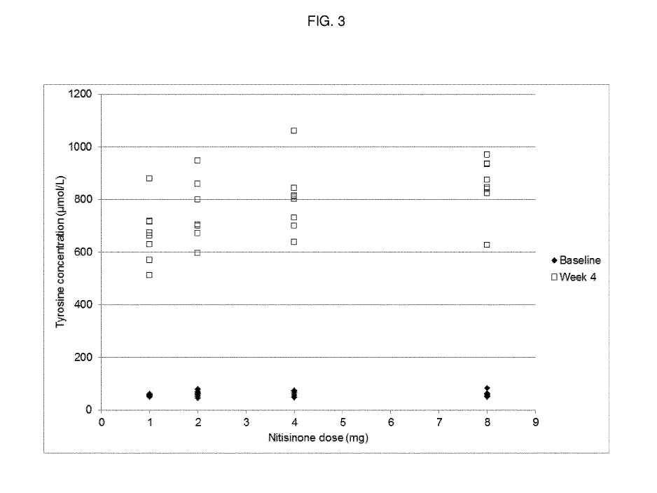

FIG. 3 shows the daily average serum concentrations of tyrosine (μmol/L) at baseline and week 4 for all doses. It can be seen that the inter-individual variability in s-Tyr is of the same magnitude for all doses.

Correlation Between u-HGA₂₄ and Serum Tyrosine

In order to illustrate how tyrosine concentrations increase when serum HGA concentrations or urinary excretion decrease, these variables are presented together in a series of figures shown below.

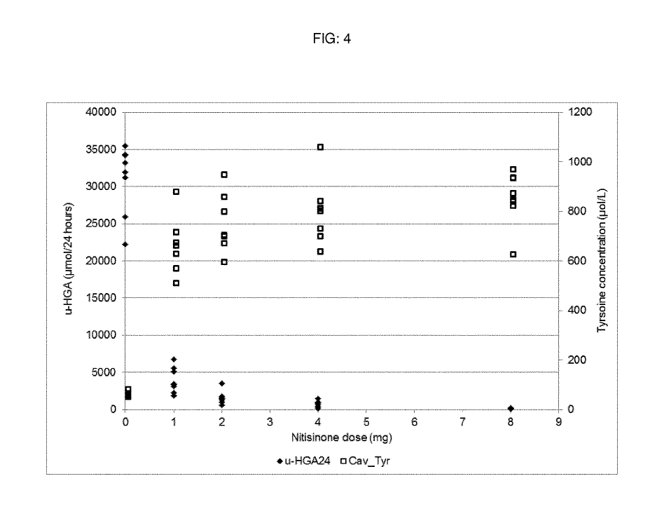

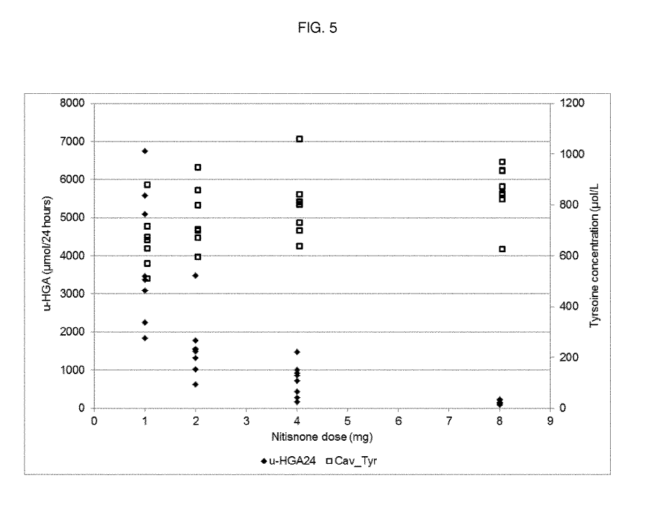

The decrease in u-HGA₂₄ and simultaneous increase in serum tyrosine are shown for all patients in FIG. 4, and, for better resolution, in FIG. 5 (nitisinone-treated patients only).

It can be seen from these figures that the urinary excretion of HGA decreases in a clear dose-related manner with increasing doses of nitisinone (up to more than >99% for the highest dose). The inter-individual variability of u-HGA₂₄ also decreases with increasing doses of nitisinone.

At the same time, the dose-response relationship for serum concentrations of tyrosine is less distinct than observed for u-HGA₂₄. There is a slight increase in mean s-Tyr with increasing doses of nitisinone, but the increase between the lowest (1 mg) and the highest dose (8 mg) is only moderate. Furthermore, the inter-individual variability in s-Tyr is of the same magnitude for all doses.

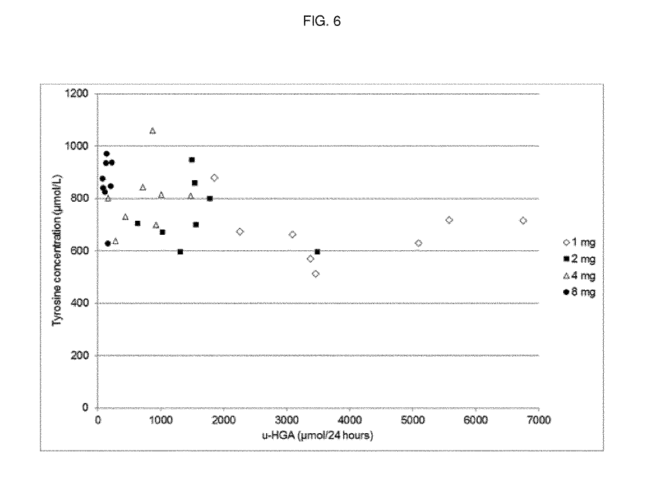

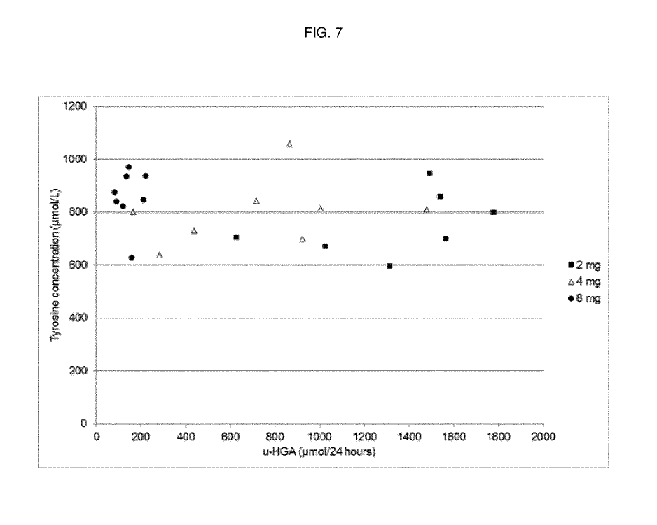

The relationship between u-HGA₂₄ and serum tyrosine is also illustrated in FIG. 6. There is no clear correlation between s-Tyr and u-HGA₂₄. However, when u HGA₂₄ is above 2000 μmol, s-Tyr is in the range of 500 to 700 μmol/L. When u-HGA₂₄ is decreased to below 2000 μmol (i.e., for most patients on doses from 2 mg and upward), then s-Tyr is in the range of 600-1000 μmol/L but with no correlation between s-Tyr and u-HGA₂₄. This is illustrated in FIG. 7, where only data for the 2-, 4- and 8-mg doses are shown (data for one patient on 2 mg, with u-HGA₂₄=3484 μmol and s-Tyr=596 μmol/L not shown).

1. A method for treatment of alkaptonuria in a human patient comprising administering nitisinone to the patient, wherein nitisinone is administered in a dose of at least 4 mg per day.

2. The method according to claim 1, wherein nitisinone is administered in a dose of at least 6 mg per day.

3. The method according to claim 1, wherein nitisinone is administered in a dose of at least 8 mg per day.

4. The method according to claim 1, wherein nitisinone is administered in a dose of at least 10 mg per day.

5. The method according to claim 1, wherein nitisinone is administered in a dose of at least 4 and up to 15 mg per day.

6. The method according to claim 1, wherein nitisinone is administered in a dose of 8-12 mg per day.

7. The method according to claim 1, wherein nitisinone is administered in a dose of 10 mg per day.

8. The method according to claim 1, wherein nitisinone is administered once daily.

9. The method according to claim 1, wherein nitisinone is administered orally.

10. The method according to claim 1, wherein nitisinone is administered as a pharmaceutical composition which comprises nitisinone in admixture with one or more pharmaceutically acceptable excipients.

11-13. (canceled)

14. The method of claim 1, wherein the patient is in need of such treatment at the recited dosage.

15. The method according to claim 2, wherein nitisinone is administered once daily.

16. The method according to claim 3, wherein nitisinone is administered once daily.

17. The method according to claim 4, wherein nitisinone is administered once daily.

18. The method according to claim 5, wherein nitisinone is administered once daily.

19. The method according to claim 6, wherein nitisinone is administered once daily.

20. The method according to claim 2, wherein nitisinone is administered as a pharmaceutical composition which comprises nitisinone in admixture with one or more pharmaceutically acceptable excipients.

21. The method according to claim 3, wherein nitisinone is administered as a pharmaceutical composition which comprises nitisinone in admixture with one or more pharmaceutically acceptable excipients.

22. The method according to claim 4, wherein nitisinone is administered as a pharmaceutical composition which comprises nitisinone in admixture with one or more pharmaceutically acceptable excipients.

23. The method according to claim 5, wherein nitisinone is administered as a pharmaceutical composition which comprises nitisinone in admixture with one or more pharmaceutically acceptable excipients.
